(19) United States
(12) Patent Application Publication
(10) Pub. No.: US 2002/0006627 A1
(43) Pub. Date: Jan. 17, 2002

(54) Method for diagnosis of Alzheimer's disease

(76) Inventors: Allen B. Reitz, Lansdale, PA (US); Adrian Dawkes, Windsor (GB); Carlos R. Plata Salaman, Ambler, PA (US); Haou-Yan Wang, Philadelphia, PA (US)

Correspondence Address: Philip S. Johnson, Esq., Johnson & Johnson, One Johnson & Johnson Plaza, New Brunswick, NJ, 08933-7003, US

(21) Appl. No.: 09/774,459

(22) Filed: Jan. 31, 2001

Related U.S. Application Data
(60) Provisional application No. 60/179,976, filed on Feb. 3, 2000.

(51) Int. Cl. G01N 33/567; G01N 33/53; G01N 33/537; G01N 33/543

(52) U.S. Cl. 435/7.21

(57) ABSTRACT

The present invention is directed to a method of diagnosing Alzheimer's disease involving analysis of a test sample in such a way that β-amyloid₁₋₄₂ or Aβ3pE is completely or nearly completely (i.e., thoroughly) dissociated from binding proteins prior to the analysis of the levels of β-amyloid₁₋₄₂ or Aβ3pE.

### CROSS-REFERENCE TO RELATED APPLICATION

This application claims priority from United States provisional application Serial No. 60/179,976, filed Feb. 3, 2000, the contents of which are hereby incorporated by reference.

### FIELD OF THE INVENTION

The present invention provides a method for diagnosing Alzheimer's disease (AD) in a subject. More particularly, a method for diagnosing AD by completely or nearly-completely releasing β-amyloid₁₋₄₂ or Aβ3pE from binding proteins in a test sample and then analyzing the quantity of elevated levels of β-amyloid₁₋₄₂ or Aβ3pE found in AD patients, or those who are in the early stages of AD, or who will develop AD in the future.

### BACKGROUND OF THE INVENTION

Neurodegenerative disorders such as Alzheimer's disease (AD) and Parkinson's disease (PD) afflict humanity with great suffering and financial loss. AD is characterized by neurofibrillary tangles, neuritic plaques, and neuronal cell death. AD appears as either the familial, early onset (<60 yrs) or late-onset (>60 yrs) forms, with the latter being more prevalent. AD is the major cause of age-related dementia and cognitive impairment (Wisniewski, T.; Ghiso, J.; Frangione, B. Neurobiol. of Disease 1997, 4, 313-328). The amyloid precursor protein (APP), β-amyloid₁₋₄₀ (Aβ₁₋₄₀), and β-amyloid₁₋₄₂ (Aβ₁₋₄₂) are keenly involved in the pathology of AD. The Aβ peptides are derived from APP by proteolytic processing. Dramatic evidence implicating the Aβ peptides, particularly Aβ₁₋₄₂, in AD comes from various recently identified mutations accounting for certain types of inherited AD. Such mutations in the presenilin (PS1 and PS2) genes are probably the cause of the most frequent form of familial, early-onset AD (Rogaev, E. I. Molecular Biology 1998, 32, 58). In these cases, as with APP mutations, more Aβ₁₋₄₂ is observed relative to Aβ₁₋₄₀. Extensive studies have shown that Aβ₁₋₄₂ has a greater ability than Aβ₁₋₄₀ to aggregate into the amyloid fibrils that constitute the plaques characteristic of AD (Lansbury, P. T., Jr. Accts. Chem. Res. 1996, 29, 317). Even though Aβ₁₋₄₀ is generally present to a much larger degree in the cerebrospinal fluid than Aβ₁₋₄₂, it is Aβ₁₋₄₂ that is present to a greater degree in AD plaques. Another major amyloid related peptide found in plaques is an N-terminally truncated variant in which the two N-terminal amino acids are removed, and glutamic acid at position three has formed a pyroglutamyl residue. (Saido, T. C.; Iwatsubo, T.; Mann, D. M. A.; Shimada, H.; Ihara, Y.; Kawashima, S. Neuron 1995, 14, 457. Iwatsubo, T.; Saido, T. C.; Mann, D. M. A.; Lee, V. M. -Y.; Trojanowski, J. Q. Am. J. Path. 1996, 149, 1823. Russo, C.; Saido, T. C.; DeBusk, L. M.; Tabaton, M.; Gambetti, P.; Teller, J. K. FEBS Lett. 1997, 409, 411.) This peptide is termed Aβ3pE-pyroglutamyl at what was previously residue 3 of either Aβ₁₋₄₀ or Aβ₁₋₄₂. In fact, some plaques may contain this particular pyroglutamyl derivative to the extent of >50% (Kuo, Y. -M.; Emmerling, M. R.; Woods, A. S.; Cotter, R. J.; Roher, A. E. Biochem. Biophys. Res. Commun. 1997, 237, 188). The Aβ3pE has an increased tendency to form β-pleated sheets and aggregate than the parent amyloid peptides (He, W.; Barrow, C. J. Biochemistry 1999, 38, 10871). Another minor, related amyloid peptide is that in which the aspartic acid at residue one is present as the isoaspartic acid.

The Aβ peptides can inhibit cholinergic neurotransmitter function independent of neurotoxicity (Auld, D. S.; Kar, S.; Quirion, R. Trends Neurosci. 1998, 21, 43). Aβ peptides bind to a number of natural substances such as apoE3, apoE4, apoJ, transthyretin, and albumin. In addition, Aβ has been reported to interact with a membrane-bound receptor for advanced glycation end products and to the class A scavenger receptor (SR) associated with the production of reactive oxygen species.

Recently, it has been described that circulating levels of β-amyloid₁₋₄₂ in plasma are elevated ca. 6× in patients with AD compared to age-matched controls (Kuo, Y. -M.; Emmerling, M. R.; Lampert, H. C.; Hempelman, S. R.; Kokjohn, T. A.; Woods, A. S.; Cotter, R. J.; Roher, A. E. Biochem. Biophys. Res. Commun. 1999, 257, 787-791).

The pretreatment of plasma with formic acid was found to be absolutely critical in dissociating the β-amyloid₁₋₄₂ from plasma proteins, prior to evaluation of the β-amyloid₁₋₄₂ levels. Without the pretreatment, levels of β-amyloid₁₋₄₂ are significantly lower. The present invention incorporates a step involving the complete or nearly-complete dissociation of β-amyloid₁₋₄₂, or related amyloid peptides such as Aβ3pE, from test samples obtained from humans, prior to analysis of β-amyloid₁₋₄₂ or Aβ3pE levels, as part of a diagnostic assay for the early detection of AD, or the propensity to develop AD in the future.

### SUMMARY OF THE INVENTION

The present invention is directed to a method of diagnosing Alzheimer's disease involving analysis of a test sample in such a way that β-amyloid₁₋₄₂ or Aβ3pE is completely or nearly completely (i.e., thoroughly) dissociated from binding proteins prior to the analysis of the levels of β-amyloid₁₋₄₂ or Aβ3pE.

We propose to diagnose patients with AD, or those who are gradually developing AD, or those who might develop AD in the future, by a procedure which involves analysis of the quantity of β-amyloid₁₋₄₂ or Aβ3pE isolated from a test sample. Those patients whose β-amyloid₁₋₄₂ or Aβ3pE levels are determined to be above a certain threshold level would be considered to either have AD or have a high probability of developing AD in the future. Further, the procedure could be done at different times, and the relative increase in the levels of β-amyloid₁₋₄₂ or Aβ3pE could be taken as a diagnostic component as to the presence of AD or the liability for developing AD in the future. Also, the relative increase in the levels of β-amyloid₁₋₄₂ or Aβ3pE could be taken as a prognostic marker and a marker to monitor the progression of the disease. Levels of β-amyloid₁₋₄₂ or Aβ3pE could be determined along with standard cognitive testing for additional assessment for prognosis and progression of the disease.

### DETAILED DESCRIPTION OF THE INVENTION

The present invention provides a method of diagnosing Alzheimer's disease in a subject in need thereof comprising:

(a) obtaining a test sample from a subject wherein the test sample contains an Aβ peptide and a binding protein;

(b) contacting the test sample with a dissociation reagent thereby thoroughly dissociating the Aβ peptide from the binding protein; and

(c) measuring the quantity of Aβ peptide in the test sample.

Alternatively, the method may be further defined by adding a step to neutralize the dissociation reagent prior to analysis. This method is useful where the assay used to measure the quantity of Aβ peptide may be affected by the presence of an active dissociation reagent. For example a highly acidic or basic dissociation reagent may alter the pH of the test sample such that an immunoassay technique is not possible. The present invention also provides a method of diagnosing Alzheimer's disease in a subject in need thereof comprising:

(a) obtaining a test sample from a subject wherein the test sample contains an Aβ peptide and a binding protein;

(b) contacting the test sample with a dissociation reagent thereby thoroughly dissociating the Aβ peptide from the binding protein;

(c) contacting the test sample with a neutralizing reagent; and

(d) measuring the quantity of Aβ peptide in the test sample.

The term “subject” as used herein, refers to an animal, preferably a mammal, most preferably a human, who has been the object of treatment, observation, biochemical screening, or experiment.

A “test sample” as used herein, refers to a biological substance that contains Aβ peptide, such as red blood cells, white blood cells, platelets, ascites, urine, saliva, olfactory neuroepithelia, skin fibroblasts, cerebrospinal fluid, and other constituents of the body that may contain the Aβ peptide. Further, a sample may be a component in a larger composition, for example in a tissue section of a biopsy, where the sample may be an unisolated fraction of biological fluid or one or more cellular subtypes amongst a field of different cell types. As used herein the term “test sample” does not include serum or plasma.

As used herein the term “Aβ peptide” is a β-amyloid peptide₁₋₄₂ or enzymatically modified β-amyloid peptide, such as where aspartic acid at position one is modified to isoaspartic acid. Particularly preferred Aβ peptides are selected from the group consisting of β-amyloid₁₋₄₂ and Aβ3pE.

As used herein the term “binding protein” is any protein to which β-amyloid peptides naturally bind, such as apoE3, apoE4, apoJ, transthyretin, and albumin. The inventors contemplate that there are cell surface receptors that also bind Aβ peptides.

The term “dissociation reagent” is a material, preferably a solution, that causes Aβ peptide to dissociate from the binding protein, and optionally causes denaturation of the binding protein. Preferred dissociation reagents include acids, including formic acid, hydrochloric acid, acetic acid, sulfuric acid, bases, including sodium hydroxide, miscible organic solvents, including ethanol, methanol, DMSO, and DMF. The dissociation reagent may also be a detergent or surfactant including Triton X-100, Tween 20, Tween 80, and sodium dodecyl sulfate (“SDS”) or a chaotropic agent including urea, and guanidinium chloride. Yet another type of dissociation reagent is a compound that inhibits β-amyloid aggregation, such as raloxifene and those compounds described in Curr. Med. Chem. 1997, 4, 159; and Exp. Opin. Ther. Pat. 1997, 7, 1115.

The term “neutralizing reagent” is a material, preferably a solution that, counteracts the dissociation reagents so that the final test sample composition is suitable for an assay to measure the dissociated Aβ peptide. Where the dissociation reagent is an acid, a suitable amount of a base, as the neutralizing reagent, may be added to the test sample to prepare it for an assay. Alternatively where the dissociation reagent is a base, a suitable amount of acid, as the neutralizing reagent, may be added to the test sample. Not all dissociation reagents will require a neutralizing reagent, including miscible organic solvents or compounds that inhibit β-amyloid aggregation

The terms “thoroughly dissociated” and/or “thoroughly dissociating” as used herein when referring to dissociation of Aβ peptide from binding protein means that the Aβ peptide is completely or nearly completely dissociated from the binding protein; that is, greater than 80% (preferably, >90%, more preferably, >95%).

The levels of Aβ peptide are measured by methods known to the art including, but not limited to, immunoassay techniques, HPLC analysis, HPLC/MS (mass spectral) analysis, MALDI/TOF (matrix-assisted laser desorption/time-of-flight) mass spectral analysis, size exclusion chromatography, thioflavin-T or Congo Red staining, or ES/MS (electrospray ionization mass spectral) analysis. By comparing the levels of β-amyloid₁₋₄₂ or Aβ3pE of the patient under investigation with a normal (control) patients, one of ordinary skill in the art can readily determine whether the patient is suffering from AD, is at risk for developing AD, or one can monitor the progression of AD. A normal (control) patient is one who is known to be free from AD or whose cognitive assessments indicate that they are not suffering from AD.

A particularly preferred assay format is the immunoassay format, wherein the Aβ peptide is isolated from the test sample using an affinity capture reagent, and is detected with an affinity label reagent, both affinity moieties being capable of simultaneous interaction with the Aβ peptide. The affinity capture or affinity label reagents comprise compounds capable of specific interaction with the Aβ peptide to the exclusion of similar compounds. These compounds include, for example, synthetic or natural amino acid polypeptides, proteins (including antibodies and derivatives thereof), small synthetic organic molecules, or deoxy- or ribonucleic acid sequences with about 20-fold or greater affinity for the Aβ peptide compared to other proteins or peptides.

The labeled compounds useful in the present invention may be labeled compounds, with means of direct detection, or may be detected by an indirect means, for example by a second labeled compound.

The phrase “labeled compound” refers to moieties capable of measurement comprising radioactive atoms, enzymes, fluorescent molecules, or alternative tags, for example biotin. Particular radioisotopes useful as a label in the present invention are ³H, ¹²⁵I, ¹³¹I, ³⁵S, ³²P, or ³³P. Particular examples of enzymes suitable for use in the present invention are horseradish peroxidase, alkaline phosphatase, or luciferase. A preferred example of a detectable label is an enzyme that cleaves a substrate to yield a chromogenic or luminescent product. Particular examples of fluorescent molecules are fluorescein (FITC), rhodamine, R-phycoerythrin (PE) or Alexa™ dyes (Molecular Probes). Direct measurement is conducted by observing the presence of the radioactive atom or flourogenic molecule, or by observation of enzymatic activity of a colorimetric or luminescent substrate.

Indirect measurement is conducted by adding an additional compound including a label to the test sample so that it can interact with the compound bound to the test sample. A well-known example is when the labeled compound comprises biotin, and a second compound comprises avidin or streptavidin and a detectable label. A second well-known example is when a first antibody is used to bind to the Aβ peptide and is detected with an anti-antibody comprising a detectable label. In this case the first antibody comprises a label in that there are specific regions capable of detection within the structure of the first antibody.

Also included in the invention is a diagnostic kit in which all of the components required for a viable diagnostic determination are packaged together sufficient for complete or nearly-complete dissociation of Aβ peptide from a test sample and subsequent analysis of the levels of the Aβ peptide such as by an ELISA (enzyme linked immunosorbant assay). The components of an immunoassay diagnostic kit include a dissociation reagent, optionally a neutralizing reagent, and an affinity capture reagent, for instance, an affinity coated resin, an affinity label reagent, and optionally immunoassay control reagents. Immunoassay control reagents are those that confirm proper function of the assay system and serve to validate interpretation of the sample. A negative control is one that yields no signal from the affinity label reagent, and is often used to determine the background noise of an assay system or used to calculate the signal to noise ratio for a positive sample using calculations well known in the art. A positive control is one that yields a signal from the affinity label reagent, and is often used to validate the assay system or to compare to a sample to interpret the status of the sample. This can be done empirically using a “yes/no” system or can be made quantitative by comparing the signal generated by control samples containing known quantities of AD peptide with a test sample.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method of diagnosing Alzheimer's disease in a subject in need thereof comprising:
(a) obtaining a test sample from a subject wherein the test sample contains an Aβ peptide and a binding protein;
(b) contacting the test sample with a dissociation reagent thereby thoroughly dissociating the Aβ peptide from the binding protein; and
(c) measuring the quantity of Aβ peptide in the test sample.

2. The method of claim 1, wherein the test sample is selected from the group consisting of red blood cells, white blood cells, platelets, ascites, urine, saliva, olfactory neuroepithelia, skin fibroblasts, and cerebrospinal fluid.

3. The method of claim 1, wherein the Aβ peptide is selected from the group consisting of β-amyloid₁₋₄₂ and Aβ3pE.

4. The method of claim 1 wherein the dissociation reagent is selected from the group consisting of formic acid, hydrochloric acid, acetic acid, sulfuric acid, sodium hydroxide, ethanol, methanol, DMSO, DMF, Triton X-100, Tween 20, Tween 80, Sodium dodecyl sulfate (SDS), and raloxifene.

5. The method of claim 1 wherein the subject is suffering from cognitive impairment and/or other clinical manifestations sufficient to warrant a possible diagnosis of Alzheimer's disease.

6. The method of claim 1 wherein the subject is suspected of developing Alzheimer's disease in the future because of family history or genetic screening.

7. The method of claim 1 wherein the subject requests a diagnostic for Alzheimer's disease, or a diagnostic is recommended by a physician, because of the subject's age, or because of suspected early signs of forgetfulness or other possible loss of cognitive function.

8. The method of claim 1 wherein the subject's relative propensity to develop full Alzheimer's disease is monitored periodically, with increasing levels of β-amyloid₁₋₄₂ or Aβ3pE over time indicating an increased propensity of developing Alzheimer's disease.

9. An immunoassay diagnostic kit useful for diagnosing Alzheimer's disease according to the method of claim 1 comprising a package containing;
(a) a dissociation reagent
(b) an affinity capture reagent, and
(c) an affinity label reagent.

10. The immunoassay diagnostic kit of claim 9 further comprising immunoassay control reagents.

11. A method of diagnosing Alzheimer's disease in a subject in need thereof comprising:
(a) obtaining a test sample from a subject wherein the test sample contains an Aβ peptide and a binding protein;
(b) contacting the test sample with a dissociation reagent thereby thoroughly dissociating the Aβ peptide from the binding protein;
(c) contacting the test sample with a neutralizing reagent; and
(d) measuring the quantity of Aβ peptide in the test sample.

12. The method of claim 11, wherein the test sample is selected from the group consisting of red blood cells, white blood cells, platelets, ascites, urine, saliva, olfactory neuroepithelia, skin fibroblasts, and cerebrospinal fluid.

13. The method of claim 11, wherein the Aβ peptide is selected from the group consisting of β-amyloid₁₋₄₂ and Aβ3pE.

14. The method of claim 11 wherein the dissociation reagent is selected from the group consisting of formic acid, hydrochloric acid, acetic acid, sulfuric acid, sodium hydroxide, ethanol, methanol, DMSO, DMF, Triton X-100, Tween 20, Tween 80, Sodium dodecyl sulfate (SDS), and raloxifene.

15. The method of claim 11 wherein the subject is suffering from cognitive impairment and/or other clinical manifestations sufficient to warrant a possible diagnosis of Alzheimer's disease.

16. The method of claim 11 wherein the subject is suspected of developing Alzheimer's disease in the future because of family history or genetic screening.

17. The method of claim 11 wherein the subject requests a diagnostic for Alzheimer's disease, or a diagnostic is recommended by a physician, because of the subject's age, or because of suspected early signs of forgetfulness or other possible loss of cognitive function.

18. The method of claim 11 wherein the subject's relative propensity to develop full Alzheimer's disease is monitored periodically, with increasing levels of β-amyloid₁₋₄₂ or Aβ3pE over time indicating an increased propensity of developing Alzheimer's disease.

19. An immunoassay diagnostic kit useful for diagnosing Alzheimer's disease according to the method of claim 11 comprising a package containing;
(a) a dissociation reagent
(b) a neutralization reagent,
(c) an affinity capture reagent,
(d) an affinity label reagent.

20. The immunoassay diagnostic kit of claim 19 further comprising immunoassay control reagents.

21. A method of diagnosing Alzheimer's disease in a subject in need thereof comprising:
(a) obtaining a test sample from a subject wherein the test sample is selected from the group consisting of red blood cells, white blood cells, platelets, ascites, urine, saliva, olfactory neuroepithelia, skin fibroblasts, and cerebrospinal fluid, and the test sample contains a binding protein and an Aβ peptide selected from the group consisting of β-amyloid₁₋₄₂ and Aβ3pE;
(b) contacting the test sample with a dissociation reagent selected from the group consisting of formic acid, hydrochloric acid, acetic acid, sulfuric acid, sodium hydroxide, ethanol, methanol, DMSO, DMF, Triton X-100, Tween 20, Tween 80, Sodium dodecyl sulfate (SDS), and raloxifene, thereby thoroughly dissociating the Aβ peptide from the binding protein; and
(c) measuring the quantity of Aβ peptide in the test sample.

22. A method of diagnosing Alzheimer's disease in a subject in need thereof comprising:
(a) obtaining a test sample from a subject wherein the test sample is selected from the group consisting of red blood cells, white blood cells, platelets, ascites, urine, saliva, olfactory neuroepithelia, skin fibroblasts, and cerebrospinal fluid, and the test sample contains a binding protein and an Aβ peptide selected from the group consisting of β-amyloid₁₋₄₂ and Aβ3pE;
(b) contacting the test sample with a dissociation reagent selected from the group consisting of formic acid, hydrochloric acid, acetic acid, sulfuric acid, sodium hydroxide, thereby thoroughly dissociating the Aβ peptide from the binding protein;
(c) contacting the test sample with a neutralizing reagent; and
(d) measuring the quantity of Aβ peptide in the test sample by immunoassay.

23. The method of claim 21 wherein the quantity of Aβ peptide is detected using a labeled compound selected from the group consisting of horseradish peroxidase, alkaline phosphatase, luciferase, fluorescein (FITC), rhodamine, R-phycoerythrin (PE), and Alexa™ dyes (Molecular Probes).
